(19) United States
(12) Patent Application Publication
(10) Pub. No.: US 2026/0188026 A1
(43) Pub. Date: Jul. 2, 2026

(54) METHOD FOR DETECTING ABNORMAL ACTIVITY OF DRIVER

(71) Applicant: MiTAC Digital Technology Corporation, Taoyuan City (TW)

(72) Inventor: Hsiang-Yu HSIEH, Taoyuan City (TW)

(21) Appl. No.: 19/436,739

(22) Filed: Dec. 30, 2025

(30) Foreign Application Priority Data
Jan. 2, 2025 (TW) 114100162
Jul. 4, 2025 (TW) 114125410

(51) Int. Cl. G06V 20/59; A61B 5/00; A61B 5/11; A61B 5/18; G06T 7/70; G06V 40/20

(52) U.S. Cl. CPC G06V 20/597; A61B 5/1114; A61B 5/1118; A61B 5/1121; A61B 5/1128; A61B 5/18; A61B 5/6893; A61B 5/726; G06T 7/70; G06V 40/20; G06T 2207/20064; G06T 2207/20084; G06T 2207/30196; G06T 2207/30268

(57) ABSTRACT

A method for detecting abnormal activity of a driver implemented by a monitoring device which includes an angle acquisition unit that obtains an angle signal during a preset time period, and a processing unit. The method includes: generating a spectrogram from the angle signal; obtaining a frequency set within the preset time period; obtaining, based on the frequency set, candidate time point(s), each corresponding to a frequency in the spectrogram that is greater than a dynamic frequency threshold; obtaining a base frequency signal based on the spectrogram; converting the base frequency signal to a base angle signal using a signal conversion technique; obtaining a peak time point that corresponds to a maximum angle value of the base angle signal within the preset time period; and obtaining an occurrence period related to an occurrence of the abnormal activity based on the peak time point and the candidate time point(s).

### CROSS-REFERENCE TO RELATED APPLICATION

This application claims priority to Taiwanese Invention patent application No. 114125410, filed on Jul. 4, 2025, and Taiwanese Invention patent application No. 114100162, filed on Jan. 2, 2025, the entire disclosures of which are incorporated by reference herein.

### FIELD

The disclosure relates to a detection method, and more particularly to a method for detecting an abnormal activity of a driver.

### BACKGROUND

A conventional in-vehicle driver monitoring system (DMS) uses a fixed, preset angle threshold value to determine whether a driver is in an abnormal driving condition. Generally, the conventional DMS considers left-right head angle (yaw) changes as a distraction behavior, and considers head pitch changes as a fatigue behavior. That is, when the driver's left-right head rotation angle (yaw) exceeds a preset threshold, the conventional DMS determines that the driver is distracted. Correspondingly, when the driver's head pitch angle exceeds another preset threshold, the conventional DMS determines that the driver is fatigued.

However, when the vehicle is moving, the quality of images captured by the conventional DMS may be affected by short-term ambient lights, or by vibrations caused by bumpy roads, which may cause the driver's head to swing or nod at multiple time points. Moreover, terminal devices disposed on a vehicle are often limited in hardware computing power or system heat dissipation, which may cause the conventional DMS to misjudge the driver's driving condition based on conventional methods that use head angle, time axis, and the preset angle threshold value as determining factors. The conventional method fails to accurately and reliably determine whether the driver's driving condition is abnormal, and frequent misjudgments on the driver's driving condition may cause a user to quickly lose trust in the conventional DMS. Furthermore, the notifications generated from misjudgments may result in wasted storage (on the device or in the cloud).

### SUMMARY

Therefore, an object of the disclosure is to provide a method for detecting an abnormal activity of a driver that can alleviate at least one of the drawbacks of the prior art.

According to the disclosure, a method for detecting an abnormal activity of a driver is implemented by an in-vehicle monitoring device. The monitoring device includes an angle acquisition unit that is configured to obtain an angle signal during a preset time period, and a processing unit that is electrically connected to the angle acquisition unit. The method comprising steps of, by the processing unit: generating a spectrogram from the angle signal by performing a time-frequency analysis; obtaining, based on a high frequency part of the spectrogram, a frequency set within the preset time period; obtaining, based on the frequency set, at least one candidate time point, each corresponding to a frequency in the spectrogram that is greater than a dynamic frequency threshold; obtaining a base frequency signal based on the spectrogram; converting the base frequency signal to a base angle signal using a signal conversion technique; obtaining a peak time point that corresponds to a maximum angle value of the base angle signal within the preset time period; and obtaining an occurrence period related to an occurrence of the abnormal activity based on the peak time point and the at least one candidate time point.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating an in-vehicle monitoring device according to an embodiment of the disclosure.

FIG. 2 is a flow chart illustrating a method for detecting an abnormal activity of a driver according to an embodiment of the disclosure.

FIG. 3 is a flow chart illustrating sub-steps for obtaining head rotation angles of the driver according to an embodiment of the disclosure.

FIG. 4 is a flow chart illustrating sub-steps for obtaining a base frequency signal according to an embodiment of the disclosure.

FIG. 5 is a flow chart illustrating sub-steps for obtaining an occurrence period related to an occurrence of the abnormal activity of the driver according to an embodiment of the disclosure.

FIG. 6 is a flow chart illustrating sub-steps for obtaining the occurrence period related to the occurrence of the abnormal activity of the driver according to another embodiment of the disclosure.

FIG. 7 is a schematic diagram illustrating a waveform plot that includes two base angle signals and two frequency sets for determining the occurrence of the abnormal activity of the driver according to one embodiment of the disclosure.

FIG. 8 is a schematic diagram illustrating another waveform plot for determining a type of abnormal activity of the driver according to one embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

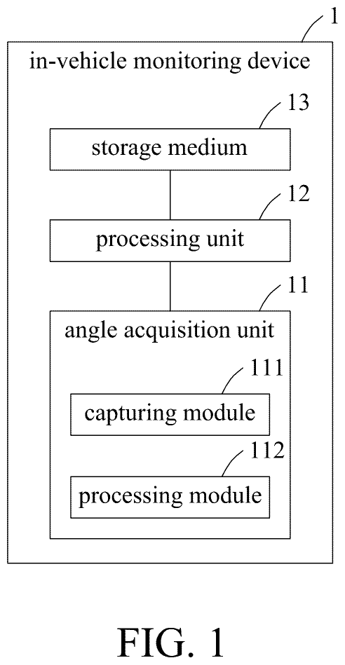

Referring to FIG. 1, according to an embodiment of the disclosure, a method for detecting an abnormal activity of a driver is implemented by an in-vehicle monitoring device 1. The monitoring device 1 includes an angle acquisition unit 11 that is configured to obtain an angle signal related to head rotation angles of the driver during a preset time period, a processing unit 12 that is electrically connected to the angle acquisition unit 11, and a storage medium 13 that is electrically connected to the processing unit 12. The angle acquisition unit 11 includes a capturing module 111 that is configured to obtain a plurality of images (or a video) related to a head portion of the driver during the preset time period, and a processing module 112 that is electrically connected to the capturing module 111 and that is configured to convert the images to the angle signal. In this embodiment, the capturing module 111 may be implemented as an onboard camera, an omnidirectional camera, or a front view camera that is configured to obtain the images. Each of the processing module 112 and the processing unit 12 may be implemented by a central processing unit (CPU), a microcontroller (MCU), a single-board computer (SBC), or an on-board computer. In some embodiments, the processing module 112 and the processing unit 12 may be integrated as one device. In one example, a length of the preset time period is two minutes, but the disclosure is not limited to such. In this embodiment, the storage medium 13 may be embodied using one or more computer-readable storage mediums such as hard disk drives or flash memory. In some embodiments, the storage medium 13 may be implemented by a cloud server connected to the processing unit 12 through a network (e.g., the Internet).

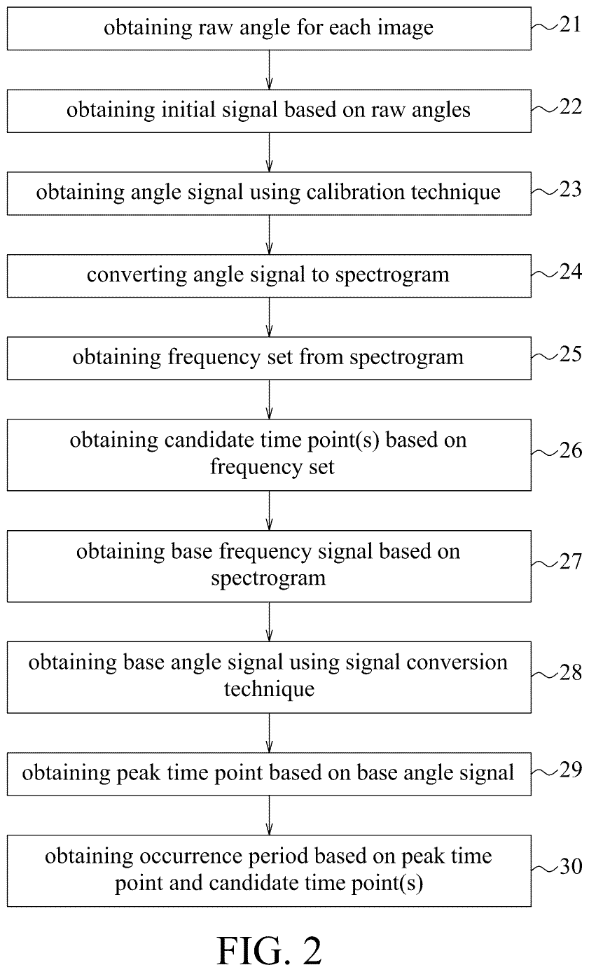

Referring further to FIG. 2, the method includes steps 21 to 30.

In step 21, for each of the images, the processing module 112 obtains a raw angle of the head portion of the driver in the image. In this embodiment, each raw angle indicates a yaw angle (i.e., an angle related to left-right rotation) of the head portion, or a pitch angle (i.e., an angle related to up-down rotation) of the head portion, but the disclosure is not limited to such.

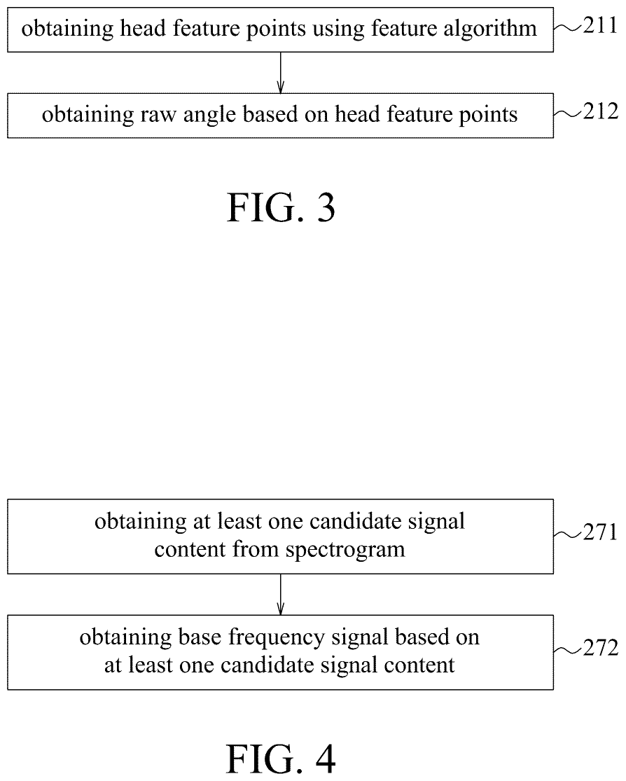

Referring further to FIG. 3, step 21 includes sub-steps 211 and 212.

In sub-step 211, for each of the images, the processing module 112 obtains a plurality of head feature points of the head portion of the driver in the image using a feature algorithm. In this embodiment, the head portion feature points include the positions of the eyes of the driver, and the feature algorithm is a convolutional neural network (CNN) that is trained to identify positions of eyes on an image, but the disclosure is not limited to such.

In sub-step 212, for each of the images, the processing module 112 first generates a 3-dimensional (3D) head model of the head portion of the driver in a virtual space based on the image (which is 2-dimensional) and the head feature points, and then obtains the raw angle based on the 3D head model. To describe in further detail, since the capturing module 111 is usually installed at an offset angle relative to the driver (instead of directly in front), and head rotation may be more accurately determined based on a front facing head portion, therefore, in this embodiment, the processing module 112 obtains a front vector of the head portion of the driver using a front vector model for calculating a front direction (i.e., a direction which the driver should be facing when driving) of the head portion. Then, the processing module 112 obtains an angle between the front vector and a head vector that is indicated by the head feature points to be the raw angle, but the disclosure is not limited to such. The front vector model may be implemented using a convolutional neural network (CNN), a generative adversarial network (GAN), a conditional GAN (CGAN), a variational autoencoder (VAE), and/or cycle-consistency constraint.

In step 22, the processing module 112 obtains an initial signal based on a plurality of the raw angles that are obtained respectively from the images during the preset time period in step 21, and based on a plurality of time points that correspond respectively to the images. That is, the initial signal represents the raw angles of the head portion of the driver at the time points of the images.

In step 23, the processing module 112 uses a calibration technique to obtain the angle signal based on the initial signal. To describe in further detail, since the capturing module 111 is usually disposed on the vehicle at a position that is not directly in front of the head portion of the driver, the raw angle that is obtained based on the image captured by the capturing module 111 for the head rotation of the driver may have an offset, and the calibration technique is used for compensating for such an offset. In this embodiment, the calibration technique may be implemented by using Python to execute an “apply_offset” function, where an average offset of the initial signal is first calculated, and then the average offset is removed from the initial signal using the calibration technique to obtain the angle signal, but the disclosure is not limited to such.

In step 24, the processing unit 12 converts the angle signal to a spectrogram related to the head rotation angles of the driver based on a time-frequency analysis. That is to say, the processing unit 12 generates the spectrogram from the angle signal by performing the time-frequency analysis on the angle signal. In this embodiment, the processing unit 12 coverts the angle signal to a 3D spectrogram using a wavelet transform, where the 3D spectrogram includes a time axis representing times of the preset time period, an angle axis indicating the head rotation angles, and a frequency axis representing frequencies. Moreover, the 3D spectrogram includes a plurality of signal contents of the angle signal, each corresponding to a different frequency and a different angle. Specifically, the spectrogram includes a high frequency part and a low frequency part (in the disclosure, the high frequency part and the low frequency part are relative to each other and their ranges may vary according to the 3D spectrogram obtained from the angle signal), and changes in the angle signal of the waveform during the preset time period at different frequency ranges (i.e., frequencies corresponding to the high frequency part and frequencies corresponding to the low frequency part) may be observed by analyzing the spectrogram. It should be noted that in wavelet transform, the time-frequency window adapts depending on frequency, that is, a width of the time-frequency window varies with frequency. To describe in further detail, at high frequencies, the time-frequency window is narrowed, which results in good time resolution; and at low frequencies, the time-frequency window is widened, which results in good frequency resolution. As such, by observing and comparing the waveform of the high frequency part of the spectrogram and the waveform of the low frequency part of the spectrogram during the preset time period, a time period of the abnormal activity (e.g., distraction or fatigue behavior) of the driver may be identified. Details of identifying the time period will be described later in the disclosure.

In this embodiment, the processing unit 12 performs the wavelet transform using a first equation (eq. 1) as follows:

$${W}_{φ} ( a , b ) = {\int }_{- ∞}^{∞} S ( t ) \cdot {ψ}^{*} ( \frac{t - b}{a} ) dt \qquad ( eq . 1 )$$

where “W_{φ}” represents the spectrogram, “a” represents a scale of the wavelet transform, “b” represents a shift parameter of the wavelet transform, “t” represents time, “S(t)” represents the angle signal, and “ψ” represents the complex conjugate of the mother wavelet

In step 25, the processing unit 12 obtains, based on the high frequency part of the spectrogram, a frequency set that is related to the head rotation angles of the driver and that is within the preset time period. Specifically step 25 includes sub-steps 251 and 252.

In sub-step 251, the processing unit 12 obtains, from the signal contents of the spectrogram, the high frequency part with a time-frequency window being smaller than a first window width threshold. The first window width threshold is related to the scale of the wavelet transform. Specifically, because of the characteristic of the wavelet transform, a small time-frequency window corresponds to high frequency content; therefore, by observing the spectrogram with the time-frequency window being smaller than the first window width threshold (e.g., 2 seconds), the high frequency part of the spectrogram may be obtained.

In sub-step 252, the processing unit 12 obtains the frequency set based on the portion of the high frequency part selected in sub-step 251. In this embodiment, the processing unit 12 generates the frequency set from the high frequency part using a second equation (eq. 2) as follows:

$${f}_{h i g h} ( t ) = arg max \vert {W}_{φ}^{h} ( a , b ) \vert \qquad ( eq . 2 )$$

where “f_{high}(t)” represents, for each time point of “t” of the preset time period, a frequency that corresponds to a maximum angle at the time point “t”,

$$“ {W}_{φ}^{h} ( a , b ) ”$$

represents the high frequency part of the spectrogram that is converted in step 24. It should be noted that, by using the second equation, the frequencies that correspond respectively to the time points when the head rotation angle of the driver is at the maximum angle in the high frequency part serve as the frequency set.

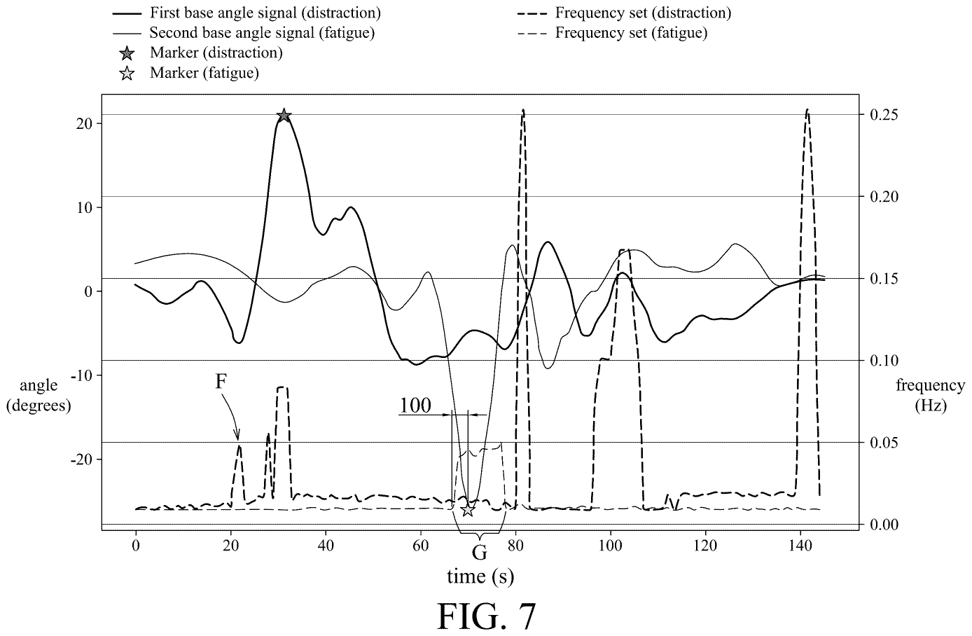

In step 26, the processing unit 12 obtains a set of candidate time points. The set of candidate time points includes at least one candidate time point each corresponding to a frequency that is in the frequency set and that is greater than a dynamic frequency threshold (e.g., 0.025 Hz), where the dynamic frequency threshold is able to effectively remove noises in the frequency set. It should be noted that, depending on a shape of the waveform of the spectrogram, each candidate time point may be obtained as an instantaneous time point, for example, when the waveform has a peak (labelled as “F” in FIG. 7) greater than the dynamic frequency threshold, or a time period, for example, during which a portion (labelled as “G” in FIG. 7) of the waveform corresponds to frequencies greater than the dynamic frequency threshold. In this embodiment, the processing unit 12 obtains the set of candidate time points using a third equation (eq. 3) as follows:

$${t}_{i} = \{ t \vert {f}_{h i g h} ( t ) \geq {f}_{t h} \vert \} , i = 1 , 2 , … , n , n \geq 1 \qquad ( eq . 3 )$$

where “tᵢ” represents the set of candidate time points, “i” represents positive integers from 1 to “n”, “f_{high}(t)” represents the frequency set obtained in step 25, and “fₜₕ” represents the dynamic frequency threshold.

In step 27, the processing unit 12 obtains a base frequency signal based on the spectrogram. It should be noted that step 27 separates the signal contents of the spectrogram, and filters out the high frequency part from the spectrogram, so as to obtain the base frequency signal from the low frequency part.

Referring further to FIG. 4, step 27 includes sub-steps 271 and 272.

In sub-step 271, the processing unit 12 obtains, from the low frequency part of the spectrogram, at least one candidate signal content, each with a time-frequency window that is greater than a second window width threshold (e.g., 8 seconds). Similar to the first window width threshold, the second window width threshold is also related to the scale of the wavelet transform.

In sub-step 272, the processing unit 12 obtains the base frequency signal based on the at least one candidate signal content. Specifically, the processing unit 12 combines the at least one candidate signal content into the base frequency signal.

It should be noted that when the head rotation angles or a pose of the driver changes substantially (e.g., distraction or fatigue behavior), a time point of such changes may be clearly indicated by peaks or valleys in the waveform of the spectrogram in the high frequency part due to the good time resolution of the high frequency part. However, the high frequency part of the spectrogram would also include false distraction or false fatigue patterns of peaks or valleys caused by shaking of the driver's head due to vibration of the vehicle during driving, or by changes in the ambient light inside or outside the vehicle. On the other hand, when the driver actually has distraction or fatigue behaviors, such behaviors may be clearly indicated by peaks or valleys in the low frequency part of the waveform of the spectrogram due to its good frequency resolution, while false distraction or false fatigue patterns as described above would not be obvious. However, a time point of the distraction or fatigue behaviors may not be clearly identified from the peaks or valleys in the low frequency part of the waveform of the spectrogram (because of the characteristic of the wavelet transform, where the low frequency part corresponds to a high time-frequency window). Therefore, by cross referencing the waveform of the spectrogram in both the high frequency part (with good time resolution) and the low frequency part (with good frequency resolution), the false distraction or false fatigue patterns may be filtered out based on the low frequency part of the waveform of the spectrogram, and a time point of the abnormal activity of the driver may be identified based on the high frequency part of the waveform of the spectrogram.

In step 28, the processing unit 12 converts the base frequency signal to rebuild a base angle signal using a signal conversion technique to represent the head rotation angles of the driver. That is, the processing unit 12 converts the base frequency signal back to the angle signal, but only with the low frequency part. In this embodiment, the signal conversion technique is an inverse wavelet transform.

In step 29, the processing unit 12 obtains a peak time point that corresponds to a maximum angle value of the base angle signal within the preset time period. Specifically, a fourth equation (eq. 4, shown in the following) is first applied to the base angle signal to obtain at least one local maximum and at least one local minimum of the base angle signal. Each local maximum or local minimum indicates a head rotation angle of the driver during the preset time period, and the plus sign (i.e., local maximum) or the minus sign (i.e., local minimum) indicates a direction (e.g., up or down, left or right) of the head rotation by the driver. The peak time point is a time point corresponding to an extremum from among the local maximum and the local minimum with the largest magnitude. In this embodiment, the processing unit 12 obtains the peak time point using a fifth equation as follows:

$${t}_{i} = \{ t \vert \frac{d S ( t )}{d t} = 0 , \frac{{d}^{2} S ( t )}{{d}^{2}} \neq 0 \} , i = 1 , 2 , … m , m \geq 1 \qquad ( eq . 4 )$$

$${t}_{o p t} = arg {max}_{{t}_{i}} \vert S ( {t}_{i} ) \vert , i = 1 , 2 , … m , m \geq 1 \qquad ( eq . 5 )$$

where “S(t)” represents the base angle signal,

$$“ \frac{d S ( t )}{d t} ”$$

represents a first derivative of the base angle signal,

$$“ \frac{{d}^{2} S ( t )}{{d}^{2}} ”$$

represents a second derivative of the base angle signal, “tᵢ” represents a set of time points that satisfies

$$\frac{d S ( t )}{d t} = 0 and \frac{{d}^{2} S ( t )}{{d}^{2}} \neq 0 ,$$

and “tₒₚₜ” represents the peak time point. It should be noted that an aim of obtaining the first derivative of the base angle signal is to identify the local minimum(s) and the local maximum(s) of the base angle signal.

It should be noted that, since a plurality of base frequency signals will be obtained respectively for the images that were obtained during the preset time period, the base angle signals obtained in step 28 for the images may be arranged in chronological order to collectively represent the preset time period. In some embodiments, the peak time point is obtained for each base angle signal. In some embodiments, one or more peak time points may be obtained from the base angle signals based on a preset angle threshold. In some embodiments, for each angle value, a peak time point may be obtained when an absolute value of the angle value is greater than a preset value that can represent the head rotation angle of the driver who is fatigued or distracted.

In step 30, the processing unit 12 obtains an occurrence period related to an occurrence of the abnormal activity of the driver based on the peak time point and the set of candidate time points.

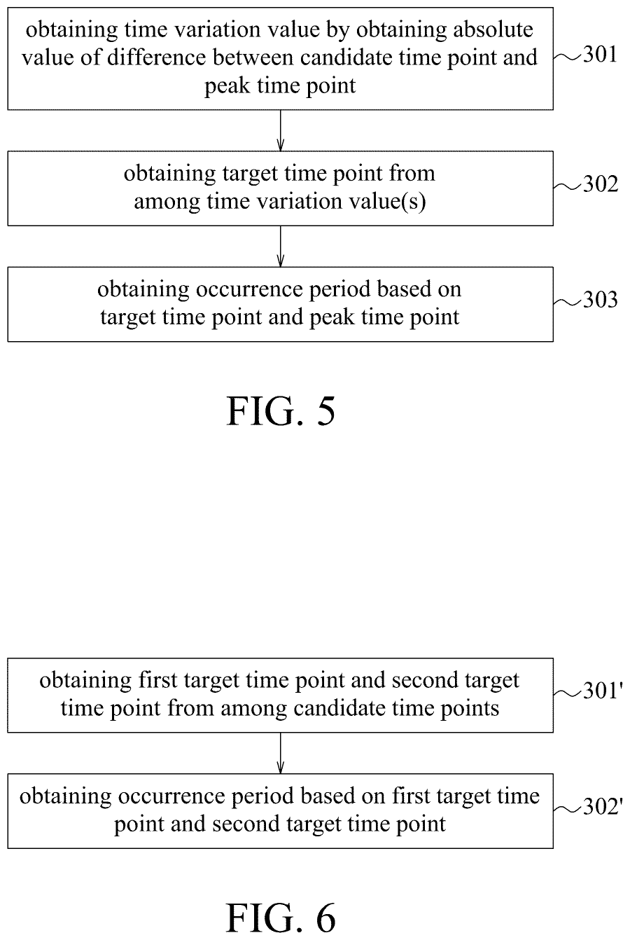

Referring further to FIGS. 5 and 7, step 30 includes sub-steps 301 to 303.

In sub-step 301, with reference to the time axis, the processing unit 12 determines, based on the frequency set, the base angle signal and the peak time point, whether the peak time point lies within the set of candidate time points obtained in step 26. That is, the processing unit 12 determines whether the peak time point corresponds to any candidate time point of the set of candidate time points (e.g., “F” or “G” in FIG. 7) or is between any two candidate time points of the set of candidate time points. When the determination is affirmative, the processing unit 12 compares the candidate time point(s) with the peak time point. Specifically, for each candidate time point included in the set of candidate time points, the processing unit 12 obtains a time variation value by obtaining an absolute value of the difference between the candidate time point and the peak time point. In this embodiment, the time variation value is equal to the absolute value of the difference between the candidate time point and the peak time point. As exemplified in FIG. 7, a first base angle signal (represented by a thick continuous line) has a star-shaped marker indicating an example peak time point related to distraction, and a second base angle signal (represented by a thin continuous line) has another star-shaped marker indicating an example peak time point related to fatigue. In some embodiments, when the peak time point lies within a set of candidate time points of a first frequency set that corresponds to pitch, and also lies within another set of candidate time points of a second frequency set that corresponds to pitch, the determination for the status of the driver may be performed for another peak time point. It is particularly noted that in some embodiments, the processing unit 12 may determine that a plurality of peak time points correspond to a plurality of candidate time points of the set of candidate time points during the preset time period, indicating that the abnormal behavior has occurred for more than one time within the preset time period.

In some embodiments, when the candidate time point is obtained as a time period instead of an instantaneous time point, the time variation value is obtained by first obtaining a representative time point from the time period, and then obtaining an absolute value of the difference between the representative time point of the candidate time point and the peak time point. In one example, the candidate time point (i.e., a time period) starts from 7 second and ends at 9 second, the representative time point of the time period may be obtained as a midpoint time of the time period (e.g., 8 second), and the time variation value is then obtained as the difference between the midpoint time of the time period and the peak time point, but the disclosure is not limited to such.

In sub-step 302, the processing unit 12 obtains, from among the time variation value(s) obtained respectively from the candidate time point(s), a target time point that corresponds to a minimum one of the time variation value(s). In this embodiment, the processing unit 12 obtains the target time point using a sixth equation as follows:

$${t}_{closest} = arg {min}_{{t}_{i}} Δ {t}_{i} , i = 1 , 2 , … , n , n \geq 1 \qquad ( eq . 6 )$$

where “t_{closest}” represents the target time point, “tᵢ” represents the candidate time point(s), and “Δtᵢ” represents the time variation value(s). In the example shown in FIG. 7, one frequency set (represented by a thin dashed line) that corresponds to the second base angle signal indicating fatigue is shown, and for each candidate time point(s) obtained for this one frequency set, a difference between the candidate time point and the peak time point (which is indicated by the star-shaped marker on the second base angle signal) is obtained, so as to obtain the target time point.

In sub-step 303, the processing unit 12 obtains the occurrence period based on the target time point and the peak time point. That is, the occurrence period is a time period between the target time point and the peak time point. In the example shown in FIG. 7, the occurrence period is labelled as a time period 100, where the starting time point of the time period 100 is the target time point, and the ending time point of the time period 100 is the peak time point.

It should be noted that, in step 26, when the set of candidate time points includes multiple candidate time points, step 30 of obtaining the occurrence period may include alternative sub-steps 301′ and 302′ (see FIG. 6) according to another embodiment.

In sub-step 301′, from among the candidate time points, the processing unit 12 obtains a first target time point that is immediately before the peak time point, and a second target time point that is immediately after the peak time point.

In one embodiment, the processing unit 12 obtains the time variation value for each candidate time point in a similar manner as described in step 301, the first target time point would be one of the candidate time points that is before the peak time point with the smallest time variation value, and the second target time point would be another one of the candidate time points that is after the peak time point with the smallest time variation value.

In sub-step 302′, the processing unit 12 obtains the occurrence period based on the first target time point and the second target time point. That is, the occurrence period is a time period between (and including) the first target time point and the second target time point.

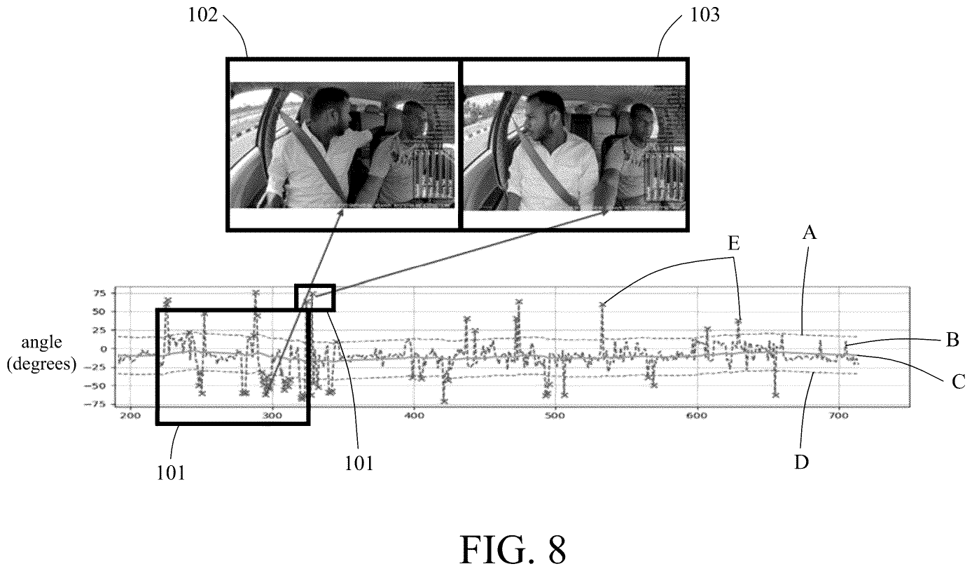

In some embodiments, the processing unit 12 may determine a type of the abnormal activity (e.g., distraction or fatigue) of the driver based on preset head rotation patterns or angle changes. To describe in further detail, a waveform plot 101 as exemplified in FIG. 8 (right-hand drive) includes a yaw angle upper threshold curve A, an actual yaw angle curve B, a base yaw angle curve C, a yaw angle lower threshold curve D and a plurality of yaw angle trigger points E. The type of the abnormal activity of the driver may be determined based on the waveform plot 101, the length of the occurrence period and the magnitude of the angle value corresponding to the peak time point, and different warning contents may be output according to the different abnormal activities. In one example, during the occurrence period between 300 and 330 seconds on the time axis of the waveform plot 101, the angle value corresponding to the peak time point is a positive value and the magnitude of the angle is greater than 45 degrees but less than 90 degrees. In such a case, the processing unit 12 determines that the driver's head is rotated to the left direction (relative to the driving direction) for 3 seconds during the occurrence period, as exemplified by a driving distraction pattern 103. In another example, during the occurrence period between 250 and 300 seconds on the time axis of the waveform plot 101, the angle value corresponding to the peak time point is a negative value and magnitude of the angle value is greater than 50 degrees but less than 75 degrees. In such a case, the processing unit 12 determines that the driver's head is rotated to the right rear direction (relative to the driving direction) for more than 3 seconds during the occurrence period, as exemplified by another driving distraction pattern 102, which shows a driver turning to the back to talk to a person sitting in the rear seat. Similarly, if the waveform plot 101 shows the driver's head pitch angle, the processing unit 12 may determine whether the driver is fatigued based on the head rotation pattern and changes in head rotation angle during the occurrence period. The preset head rotation patterns or angle changes may be adjusted based on the actual situation. Moreover, the processing unit 12 may adjust the warning contents (including emphasis and tones) based on a level of distraction or fatigue of the driver.

It should be noted that distraction and fatigue behaviors are reflected differently in the driver's head rotation pattern. For example, a pitch changing pattern from the driver being fatigued (e.g., from nodding or dozing off) would look different from a pitch changing pattern from the driver being distracted (e.g., looking down at the phone). Moreover, the frequencies of occurrence of the pitch changing pattern would also be different from the frequencies of occurrence of distraction and fatigue behaviors. For example, when the driver dozes off, the pitch angle would change repeatedly within a period of time at a relatively stable rhythm. On the contrary, when the driver is looking down at the phone, the pitch angle would change randomly at an irregular frequency. Additionally, the magnitude of pitch angle changes may be used to distinguish a level of the driver's distraction or fatigue behavior. Without further differentiation, fatigue behaviors may be incorrectly identified as distraction behaviors, resulting in incorrect driving record. Therefore, in some embodiments, the processing unit 12 is further configured to differentiate between fatigue behaviors and distraction behaviors.

To describe in further detail, the method further includes steps 31 and 32 after step 30. In step 31, the processing unit 12 determines whether there is a change in pitch (i.e., the pitch angle of the head portion changes) in the base angle signal. When determining that there is a change in pitch in the base angle signal, the flow proceeds to step 32.

In step 32, the processing unit 12 determines whether the change in pitch in the base angle signal during a determination time period conforms with a preset waveform. In some embodiments, the storage medium 13 may store a plurality of preset waveforms that are related to distraction or fatigue behavior and that are used as the basis for the processing unit 12 to make the determination in step 32. If the change in pitch in the base angle signal conforms with one of the preset waveforms that corresponds to dozing off, then the processing unit 12 determines that the type of the abnormal activity of the driver is fatigue; if the change in pitch in the base angle signal conforms with another one of the preset waveforms that corresponds to lowering the head to look at the phone, then the processing unit 12 determines that the type of the abnormal activity of the driver is distraction. It should be noted that the determination time period includes the target time point and the peak time point, and is, for example, 30 seconds; that is, the determination time period covers the occurrence period. In a first example, the preset waveform may be that the change in pitch (head portion moving up and down) occurs at similar intervals for at least three times during the determination time period. For the first example, when determining that the change in pitch in the base angle signal during the determination time period conforms with the preset waveform, the processing unit 12 determines that the type of the abnormal activity of the driver is fatigue; otherwise, the processing unit 12 determines that the type of the abnormal activity of the driver is distraction. In a second example, the preset waveform may be that the change in pitch is steep (e.g., a slope of the increase/decrease in pitch is greater than a slope threshold). For the second example, when determining that the change in pitch in the base angle signal during the determination time period conforms with the preset waveform, the processing unit 12 determines that the type of the abnormal activity of the driver is distraction.

In some embodiments, after the processing unit 12 obtains the occurrence period, the processing unit 12 may store a portion of the images (e.g., a clip of a video recorded by the in-vehicle monitoring device 1 during the preset time period) in the storage medium 13, where the portion of the images are obtained at least during the occurrence period. In one example, a saved length of the clip may be 8 seconds long, while the occurrence period may be shorter than or equal to the saved length of the clip, but the disclosure is not limited to such. The processing unit 12 may further determine whether to dynamically adjust the saved length of the clip based on the level of number of occurrences of the driver's distraction or fatigue behavior. For example, when the processing unit 12 determines that a number of instances of the occurrence of the abnormal activity during a predetermined period (one minute) is greater than a predetermined value (3 times) (i.e., the driver shows abnormal activity frequently), the processing unit 12 may increase an amount of the portion of the images that are to be stored in the storage medium 13, that is, to extend the saved length of the clip (e.g., from 8 seconds to 15 seconds). Conversely, when the number of occurrences of the abnormal activity decreases to normal level, the saved length of the clip may be reverted back to the original length (e.g., 8 seconds). As such, by extending the saved length, the clip may be used for better assessment of the driver's overall condition.

In some embodiments, when steps 31 and 32 are performed, in step 32, the processing unit 12 may further generate a marker to indicate the type of the abnormal activity of the driver. In one example, when the processing unit 12 determines, in step 32, that the change in pitch indicates fatigue, the processing unit 12 generates a fatigue marker for the clip that includes the determination time period. In another example, when the processing unit 12 determines, in step 32, that the change in pitch indicates distraction, the processing unit 12 generates a distraction marker for the clip that includes the determination time period.

In this embodiment, steps 25 and 26 are performed before steps 27 to 29, but in some other embodiments, steps 25 and 26 may be performed after steps 27 to 29, as long as they are performed before step 30. It should be noted that the order of performing the method is not limited to the abovementioned example, as long as the method under such adjustment achieves substantially the same function in substantially the same way as provided in the embodiment.

In summary, according to the disclosure, the processing unit 12 obtains the angle signal related to head rotation angles of the driver based on the images captured by the capturing module 111, and analyzes the spectrogram obtained based on the angle signal, so as to obtain the frequency set and the base frequency signal. Then, the processing unit 12 cross references the frequency set, which corresponds to the high frequency part with a relatively smaller time-frequency window, and the base frequency signal, which corresponds to the low frequency part with a relatively greater time-frequency window, so as to obtain the set of candidate time points and the peak time point, where the occurrence period of the abnormal activity of the driver may be obtained based on the set of candidate time points and the peak time point. Moreover, the processing unit 12 may store the images (or video) captured over a preset time period (e.g., 8 or 15 seconds) that are related to the occurrence of the abnormal activity of the driver. As such, instead of identifying the driver as having an abnormal activity every time the head rotation angle is greater than a threshold value at a single time point, the disclosure is able to more accurately determine whether the driver has an abnormal activity by cross referencing the waveform of the spectrogram in both the high frequency part and the low frequency part, and is able to further identify the type of abnormal activity based on the sign of the head rotation angle and the length of the occurrence period.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for detecting an abnormal activity of a driver, the method to be implemented by an in-vehicle monitoring device, the monitoring device including an angle acquisition unit that is configured to obtain an angle signal during a preset time period, and a processing unit that is electrically connected to the angle acquisition unit, the method comprising steps of, by the processing unit:
generating a spectrogram from the angle signal by performing a time-frequency analysis;
obtaining, based on a high frequency part of the spectrogram, a frequency set within the preset time period;
obtaining, based on the frequency set, at least one candidate time point, each corresponding to a frequency in the spectrogram that is greater than a dynamic frequency threshold;
obtaining a base frequency signal based on the spectrogram;
converting the base frequency signal to a base angle signal using a signal conversion technique;
obtaining a peak time point that corresponds to a maximum angle value of the base angle signal within the preset time period; and
obtaining an occurrence period related to an occurrence of the abnormal activity based on the peak time point and the at least one candidate time point.

2. The method as claimed in claim 1, wherein the step of obtaining the base frequency signal includes:
obtaining, from the spectrogram, at least one candidate signal content with a time-frequency window that is greater than a window width threshold; and
obtaining the base frequency signal based on the at least one candidate signal content.

3. The method as claimed in claim 1, the angle acquisition unit including a capturing module that is configured to obtain a plurality of images related to a head portion of the driver during the preset time period, and a processing module that is electrically connected to the capturing module and that is configured to convert the plurality of images to the angle signal, the method further comprising, before the step of generating the spectrogram, steps of:
for each image of the plurality of images, the processing module obtaining a raw angle of the head portion of the driver in the image;
the processing module obtaining an initial signal based on a plurality of the raw angles that are obtained respectively from the plurality of images during the preset time period; and
the processing module obtaining the angle signal based on the initial signal using a calibration technique.

4. The method as claimed in claim 3, wherein the step of obtaining the raw angle for each image of the plurality of images includes:
obtaining a plurality of head feature points of the head portion of the driver in the image using a feature algorithm; and
obtaining the raw angle related to the head rotation of the driver based on the plurality of head feature points.

5. The method as claimed in claim 3, wherein the calibration technique includes calculating an average offset of the initial signal, and removing the average offset from the initial signal so as to obtain the angle signal.

6. The method as claimed in claim 1, wherein, in the step of generating the spectrogram from the angle signal, the processing unit generates the spectrogram using a wavelet transform, and, in the step of converting the base frequency signal to the base angle signal, the processing unit converts the base frequency signal to the base angle signal using an inverse wavelet transform.

7. The method as claimed in claim 1, wherein the step of obtaining the occurrence period includes:
for each candidate time point of the at least one candidate time point, obtain a time variation value by obtaining an absolute value of the difference between the candidate time point and the peak time point;
from among at least one time variation value obtained respectively from the at least one candidate time point, obtaining a target time point that corresponds to a minimum one of the at least one time variation value; and
obtaining the occurrence period based on the target time point and the peak time point.

8. The method as claimed in claim 1, wherein, in the step of obtaining the at least one candidate time point, the at least one candidate time point includes a plurality of candidate time points, and the step of obtaining the occurrence period includes:
from among the plurality of candidate time points, obtaining a first target time point that is immediately before the peak time point and a second target time point that is immediately after the peak time point; and
obtaining the occurrence period based on the first target time point and the second target time point.

9. The method as claimed in claim 1, wherein the at least one candidate time point is one of an instantaneous time point and a time period.

10. The method as claimed in claim 1, the monitoring device further including a storage medium, the angle acquisition unit including a capturing module that is configured to obtain a plurality of images related to a head portion of the driver during the preset time period,
the method further comprising, after obtaining the occurrence period, storing a portion of the plurality of images in the storage medium, where the portion of the plurality of images are obtained during the occurrence period.

11. The method as claimed in claim 10, further comprising, after obtaining the occurrence period:
determining whether a number of instances of the occurrence of the abnormal activity during a predetermined period is greater than a predetermined value; and
in response to determining that the number of instances of the occurrence of the abnormal activity during the predetermined period is greater than the predetermined value, increasing an amount of the portion of the plurality of images that are stored in the storage medium.

12. The method as claimed in claim 11, further comprising:
in response to determining that the number of instances of the occurrence of the abnormal activity during the predetermined period is greater than the predetermined value, determining a type of the abnormal activity based on a portion of the base angle signal that corresponds to the occurrence period and a plurality of preset head rotation patterns;
in response to determining that the type of the abnormal activity is distraction, generating a distraction marker for the portion of the plurality of images; and
in response to determining that the type of the abnormal activity is fatigue, generating a fatigue marker for the portion of the plurality of images.

13. The method as claimed in claim 10, further comprising, after obtaining the occurrence period:
determining whether a change in the base angle signal during a determination time period conforms with a preset waveform;
in response to determining that the change in the base angle signal conforms with the preset waveform, determining that a type of the abnormal activity of the driver is one of fatigue and distraction.
